(19) United States
(12) Patent Application Publication
(10) Pub. No.: US 2003/0133831 A1
(43) Pub. Date: Jul. 17, 2003

(54) Method of impregnating polymeric medical devices with triclosan

(76) Inventors: Christopher J. Knors, Raleigh, NC (US); Yelena Tropsha, Chapel Hill, NC (US); Noel G. Harvey, Efland, NC (US); Richard P. Clarke, Raleigh, NC (US)

Correspondence Address: BECTON, DICKINSON AND COMPANY, 1 BECTON DRIVE, FRANKLIN LAKES, NJ, 07417-1880, US

(21) Appl. No.: 10/354,620

(22) Filed: Jan. 30, 2003

Related U.S. Application Data
(63) Continuation-in-part of application No. 09/451,831, filed on Nov. 30, 1999, now abandoned.

(51) Int. Cl. A61L 9/00; B01J 19/00; B01D 11/04; B01D 11/02; A61L 2/00

(52) U.S. Cl. 422/28; 422/1; 422/32; 422/33; 422/40; 422/256; 422/261; 422/295; 422/300; 422/307

(57) ABSTRACT

A method of impregnating a polymeric medical device with an antimicrobial agent is disclosed. The method involves forming a solution by dissolving triclosan in a compressed fluid and contacting the polymeric medical device with the solution. After the solution has been infused into the polymeric medical device, the solution and the medical device are separated.

### FIELD OF THE INVENTION

The present invention relates to impregnating medical devices with an antimicrobial agent. More particularly, the present invention relates to a method of impregnating polymeric medical devices with an antimicrobial agent utilizing a compressed fluid and products derived from the method, wherein the impregnation process optimizes release of the antimicrobial agent for use with medical devices.

### BACKGROUND OF THE INVENTION

Medical devices contaminated with pathogens can infect persons who contact the contaminated device. Such infections are a frequent complication during use of medical devices which contact body tissue or fluid. Catheters used for vascular access, abdominal cavity tubing, drainage bags, and various connectors used in conjunction with such devices are common sources of infection. In particular, a high percentage of patients who require long-term urinary catheters develop chronic urinary tract infections, frequently in conjunction with episodes of fever, chills, and flank pain.

Therefore, it is desirable to provide a medical device having infection controlling properties. Medical articles are frequently fabricated from polymeric materials such as silicone rubber, ABS, or polyurethane by molding and extruding techniques. One method of imparting antimicrobial properties to medical devices made from polymeric materials is to incorporate an antimicrobial agent into the material during the process of forming the device. However, these forming processes generally involve high temperatures that have a tendency to decompose many antimicrobial agents. This decomposition during the forming process requires incorporating more agent in the composition than is actually required, which increases manufacturing cost of the device. These decomposition products may render the device unsuitable for use due to discoloration or toxicity.

Another problem associated with incorporating the antimicrobial agent into the material during the forming process is that some antimicrobial agents tend to interfere with crosslinking. Such interference may prevent proper formation of the polymeric based material for the medical device. This may result in the medical device having undesirable qualities. More importantly, incorporation of antimicrobial agents during melt processing results in a homogenous distribution of agent throughout the plastic device. Homogenous distribution of antimicrobial agents minimizes the total amount of agent released during use of the device.

Another way to incorporate an antimicrobial agent into a polymeric medical device is to soak the device in a solution of a volatile solvent and an antimicrobial agent. For example, U.S. Pat. No. 5,772,640, to Modak et al., discloses dissolving a combination of chlorhexidene compounds and triclosan in solvents such as methanol, ethanol, and hexane, and soaking the medical device in the solution. It should be appreciated that the medical device must have some affinity for the solvent used, such that the solvent can penetrate the plastic device along with the antimicrobial agent, and the agent must be soluble in the solvent. However, one problem with this method is that it is difficult to completely remove the solvent from the polymer. Minor amounts of residual solvent left in the medical device may be problematic, especially if the solvent is toxic. Residual solvent may also undesirably change the physical properties of the device, for example, loss of physical shape or dimension by swelling, or loss of tensile properties. In addition, many of these solvents are flammable.

It would be desirable to provide a method for infusing an antimicrobial agent into a medical device that did not require high temperature processing or solvents that are difficult to remove from the polymer material. It would also be useful to provide a method for impregnating a medical device with an antimicrobial agent that did not require the use of flammable or toxic solvents that remain in the material after the impregnation process. It would also be useful to provide a method for impregnating a medical device with an antimicrobial agent so that the agent is non-homogeneously dispersed in the device. In particular, it would be useful to have a greater concentration of the agent at the surface of the device exposed to bodily fluids compared to the underlying non-exposed surface to provide optimal release of the agent during use.

### SUMMARY OF THE INVENTION

The present invention generally provides a method of impregnating a polymeric medical device with an antimicrobial agent. In a preferred embodiment, the agent is non-homogeneously dispersed within the device. In one preferred embodiment, the concentration of the antimicrobial agent decreases from the exterior of the device to the interior of the device.

The method comprises dissolving triclosan in a compressed fluid to form a solution and contacting the polymeric medical device with the solution at a pressure and temperature and for a time sufficient to diffuse the solution into the polymeric medical device. After the solution has diffused into the polymeric medical device, the solution is separated from the polymeric medical device by decompressing the fluid.

Preferably, the compressed fluid is carbon dioxide. In one embodiment, the pressure of the solution is maintained above 1500 pounds per square inch during the contacting step. In another aspect, the present invention includes medical devices made by the method of the invention. Such medical devices include, but are not limited to, catheters and catheter connectors.

The present invention provides a method of impregnating an antimicrobial agent into a polymeric medical device such that the agent is non-homogeneously dispersed within the device. Advantageously, the impregnation process can be performed at a temperature that does not deteriorate the antimicrobial agent. Another advantage of the present invention is that flammable and toxic solvents are not utilized during the impregnation process. Additional features and advantages of the invention will be set forth in the following description.

### BRIEF DESCRIPTION OF THE DRAWINGS

FIG. 1 is a graph showing FT-IR data of silicone rubber that has been non-homogeneously impregnated with triclosan using the method of the invention.

FIG. 2 is a graph showing FT-IR data of silicone rubber homogeneously impregnated with triclosan by melt blending.

FIG. 3 is a graph showing comparative triclosan elution data of melt blended samples and samples made by the method of the invention soaked in aqueous media at 25° C.

FIG. 4 is a graph comparing elution data of melt blended samples and samples made by the method of the invention soaked in aqueous media at 38° C.

### DETAILED DESCRIPTION

According to the method of the invention, a compressed fluid is used to impregnate polymeric medical devices with triclosan. After extensive experimentation with various antimicrobial agents, applicants have discovered that triclosan was the only agent tested that could be impregnated into a polymeric medical device utilizing a compressed fluid.

A compressed fluid is a dense gas that is maintained at or near its critical temperature (T_{c}), wherein T_{c} is the temperature above which it cannot be liquefied by pressure. When compounds such as CO₂, N₂O, ethylene, and ethane are maintained at or above their critical temperatures and pressures, the compounds form supercritical fluids. Compressed fluids can serve dual functions as a solvent for an antimicrobial agent impregnated into a medical device and as a swelling agent for a polymeric material. The preferred compressed fluid of the present invention is carbon dioxide.

According to the present invention, triclosan and a polymeric medical device are placed in a pressure vessel and charged with carbon dioxide at a pressure exceeding 800 pounds per square inch and at a temperature exceeding 0° C. In a preferred embodiment, the temperature range is between about 0° C. 50° C. and the pressure is in the range of about 850 psi to about 5000 psi. The pressure vessel is held at or below supercritical conditions for a time sufficient to dissolve the triclosan in the compressed fluid and to infuse the triclosan to the desired depth into the medical device. The solution of triclosan and compressed fluid is separated from the medical device by slowly venting the pressure in the vessel. Excessive rapid venting results in foaming of the device surface.

In the following examples, experiments for infusing various antimicrobial additives into polymer substrates were conducted in small, high pressure reactors. The reactors were of similar design with configurations allowing variable displacement. The reactors used in these examples had displacements of 2 ml and 4 ml. The reactors were constructed of stainless steel compression fittings coupled onto stainless steel bodies. The body dimensions were ½ inch in diameter×1 inch in length for the 4 ml reactor and ½ inch in diameter×1/2 inch in length for the 2 ml reactor. The reactor was equipped with a single valve for loading and discharging carbon dioxide. The valve could be removed entirely for the addition of antimicrobial additives and polymer samples. For comparative examples, antimicrobial agent was melt blended into polymer substrates using conventional melt processing equipment well know in the art.

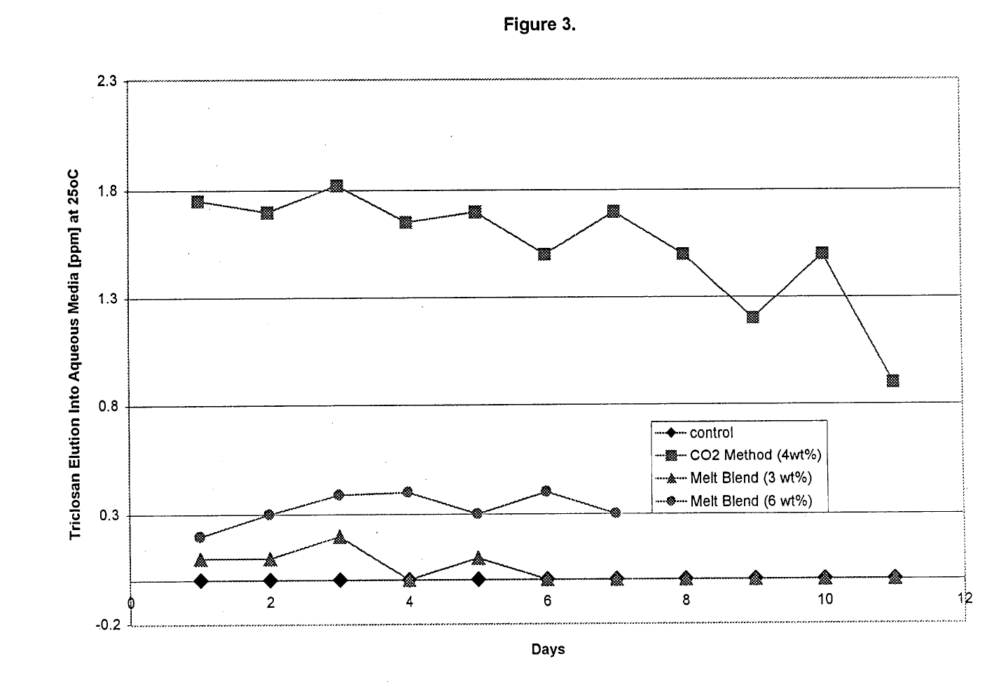

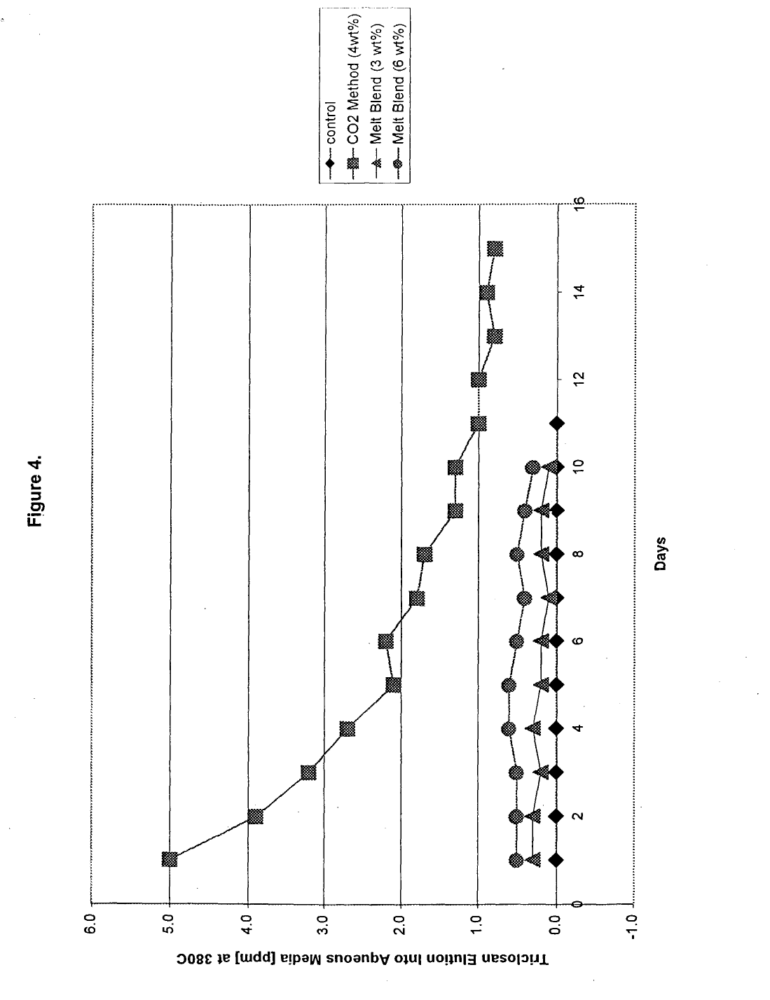

In a typical experimental run, 100 mg of solid antimicrobial was loaded into the reactor along with one to three polymer samples. Polymer samples were usually ⅜ inch diameter discs punched from sheet stock. When multiple discs were treated during a single run, the discs were separated by stainless steel mesh, to allow full exposure to the reaction mixture. The reactor was sealed and liquid carbon dioxide and charged to about 1800 pounds per square inch (psi). The antimicrobial additive and polymer sample were exposed to the liquid carbon dioxide for a period ranging from a few minutes to a few days, allowing the antimicrobial agent to dissolve and diffuse into the polymer. The carbon dioxide and antimicrobial agent solution was separated from the polymeric sample by slowly venting the carbon dioxide, and the polymer samples were recovered and examined for weight change, or change in infrared spectra. The samples made in example IV and comparative examples X-XII were then exposed to aqueous media, and the release of triclosan from the polymeric samples was monitored over a period of days using high pressure liquid chromatography (HPLC). The triclosan release data is shown in FIGS. 3 and 4.

The examples and comparative examples demonstrate that triclosan was the only antimicrobial agent of the agents tested that could be infused into the polymeric samples. In addition, polymer substrates prepared by infusion of triclosan by carbon dioxide were demonstrated to release triclosan at higher levels and for longer duration when exposed to aqueous media.

The method of the present invention is illustrated by the following examples:

### EXAMPLE I

130 mg of triclosan was loaded into a 2 ml reactor along with a single silicone rubber disc. The reactor was charged with approximately 1.6 g carbon dioxide at 1800 psi. The reactor was held at room temperature for 45 minutes, and then slowly vented. The silicone rubber disc was recovered and rinsed with water. The disc was allowed to degas overnight, and after degassing, the disc was weighed. The weight before treatment was 0.1749 g and after treatment and degassing, the weight was 0.1730 g, for a weight loss of about 2 mg. A small amount of oil was present in the reactor after treatment, presumably from extraction of uncured silicone monomer by the liquid carbon dioxide. The discs were examined using photoacoustic infrared analysis. The discs showed the presence of triclosan at or near the surface of the sample, evidenced by absorption bands in the 1300-1700 cm⁻¹ and 3000-3500 cm⁻¹ regions.

### EXAMPLE II

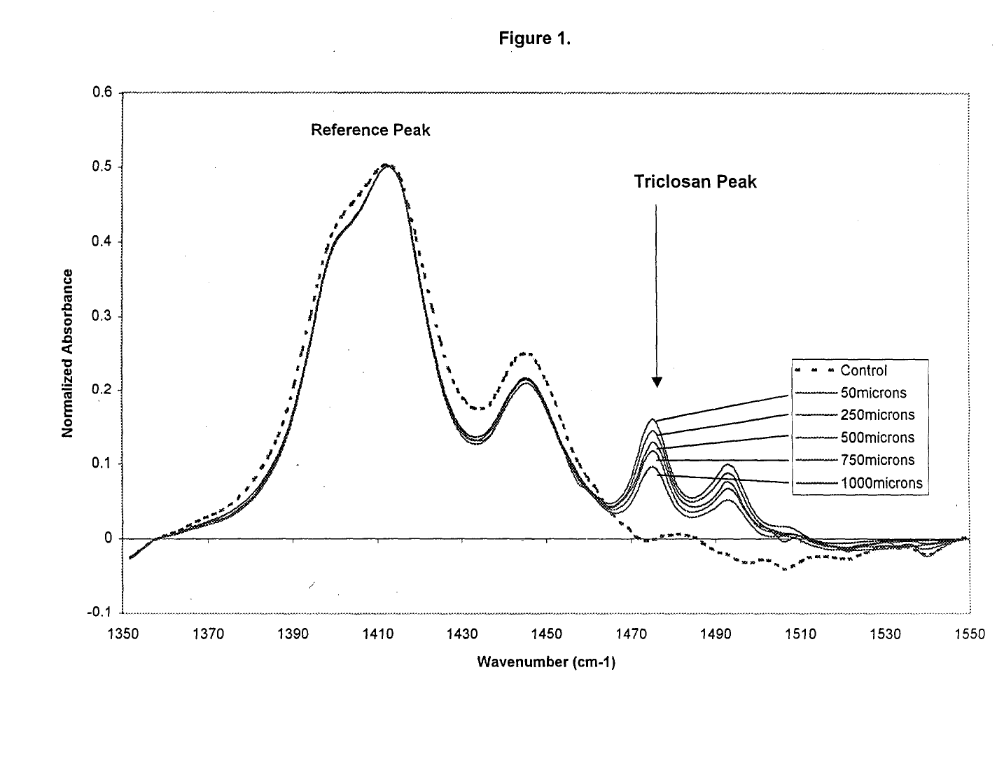

100 mg of triclosan was loaded into a 4 ml reactor along with three silicone rubber discs. The reactor was charged with approximately 3 g of carbon dioxide at 1800 psi. The reactor was held at room temperature for 1 hour, and after 1 hour, the reactor was slowly vented. The silicone rubber discs were recovered and rinsed with a 50/50 solution of MeOH/H₂O. Samples were microtomed into 0.002 inch thick sections and analyzed by transmission infrared spectroscopy. An absorption band observed at 1475 cm⁻¹ indicated the presence of triclosan. FT-IR data for this sample, shown in FIG. 1, clearly demonstrates the non-homogeneity of the triclosan agent distributed within the polymer matrix.

### EXAMPLE III

69 mg of triclosan was loaded into a 2 ml reactor along with an ABS disc. 1.6 g of carbon dioxide was charged at 1800 psi, held at room temperature for 1 hour, and then vented. The disc was recovered and allowed to degas for 5 days. The ABS sample weighed 171 mg after treatment, and 166 mg before treatment, representing a weight gain of 5 mg due to triclosan absorption.

### EXAMPLE IV

120 mg of triclosan was loaded into a 4 ml reactor along with six Vialon™ polyurethane discs. Approximately 2 g of carbon dioxide was charged at 1800 psi, stirred at room temperature for 1 hour, and then vented. The discs were recovered and allowed to degas for 24 hours. The Vialon™ discs each gained 6-7 mg in weight, representing absorption of triclosan, and the finished samples contained 4 weight percent of triclosan. Elution of triclosan from this sample in aqueous media is shown in FIGS. 3 and 4.

### EXAMPLE V

119 mg of triclosan was loaded into a 4 ml reactor along with three Elastollan™ (available from BASF) polyurethane discs. Approximately 2 g of carbon dioxide was charged at 1800 psi, and stirred at room temperature for 70 minutes, and then vented. The discs were recovered and degassed overnight. The average weight gain of each disc was about 20 mg, or about 14%, due to triclosan absorption.

### Comparative Example I

100 mg of chlorhexidine biguanide was loaded into a 2 ml reactor along with a silicone rubber disc. 1.6 g of carbon dioxide was charged at 1800 psi and allowed to sit at room temperature for 30 minutes, then vented. The disc was recovered and allowed to degas. The weight change of the disc was from 0.174 g before treatment to 0.170 g after treatment. The disc was clear and appeared to have no chlorhexidine present. The weight loss of this sample was similar to a sample treated with carbon dioxide alone.

### Comparative Example II

110 mg of chlorhexidine diacetate was loaded into a 4 ml reactor along with three silicone rubber discs. The reactor was charged with approximately 3 g of carbon dioxide at 1800 psi. The reactor was allowed to set at room temperature for 6 days, after which it was slowly vented. The silicone rubber discs were recovered and rinsed with water. Samples were microtomed into 0.002 inch thick sections and analyzed by transmission IR spectroscopy. IR spectra showed no evidence of chlorhexidine diacetate. Spectra were identical to silicone rubber.

### Comparative Example III

101 mg of silver sulfadiazine was loaded into a 2 ml reactor along with a silicone rubber disc. 1.6 g of carbon dioxide was charged at 1800 psi and allowed to sit at room temperature for 65 hours. The vessel was then vented. The disc was recovered and allowed to degas The sample was microtomed into 0.002 inch thick sections and analyzed by transmission IR spectroscopy. IR spectra showed no evidence of silver sulfadiazine. Spectrum was identical to silicone rubber.

### Comparative Example IV

53 mg of alexidine HCl was loaded into a 2 ml reactor along with two silicone rubber discs. 1.6 g of carbon dioxide was charged at 1800 psi, held at room temperature for 19 hours, and then vented. The discs were recovered and allowed to degas. Samples were microtomed into 0.002 inch thick sections and analyzed by transmission IR spectroscopy. IR spectra showed no evidence of alexidine HCl. Spectra were identical to silicone rubber.

### Comparative Example V

97 mg of benzalkonium chloride was loaded into a 2 ml reactor along with a silicone rubber disc. 1.6 g of carbon dioxide was charged at 1800 psi, held at room temperature for 1.3 hours, and then vented. The disc was recovered and allowed to degas. The sample was microtomed into 0.002 inch thick sections and analyzed by transmission IR spectroscopy. IR spectra showed no evidence of benzalkonium chloride. The spectrum was identical to silicone rubber.

### Comparative Example VI

80 mg of triclocarban was loaded into a 2 ml reactor along with a silicone rubber disc. 1.6 g of carbon dioxide was charged at 1800 psi, held at room temperature for 5 hours, and then vented. The disc was recovered and allowed to degas. The sample was microtomed into 0.002 inch thick sections and analyzed by transmission IR spectroscopy. IR spectra showed no evidence of triclocarban. Spectrum was identical to silicone rubber.

### Comparative Example VII

1 ml of chlorhexidine digluconate was loaded into a 2 ml reactor along with a silicone rubber disc. 0.8 g of carbon dioxide was charged at 1800 psi and heated to 42° C. The reactor was stirred for 21 hours, cooled and then vented. The disc was recovered and allowed to degas. The sample was microtomed into 0.002 inch thick sections and analyzed by transmission IR spectroscopy. IR spectra showed no evidence of chlorhexidine digluconate. Spectrum was identical to silicone rubber.

### Comparative Example VIII

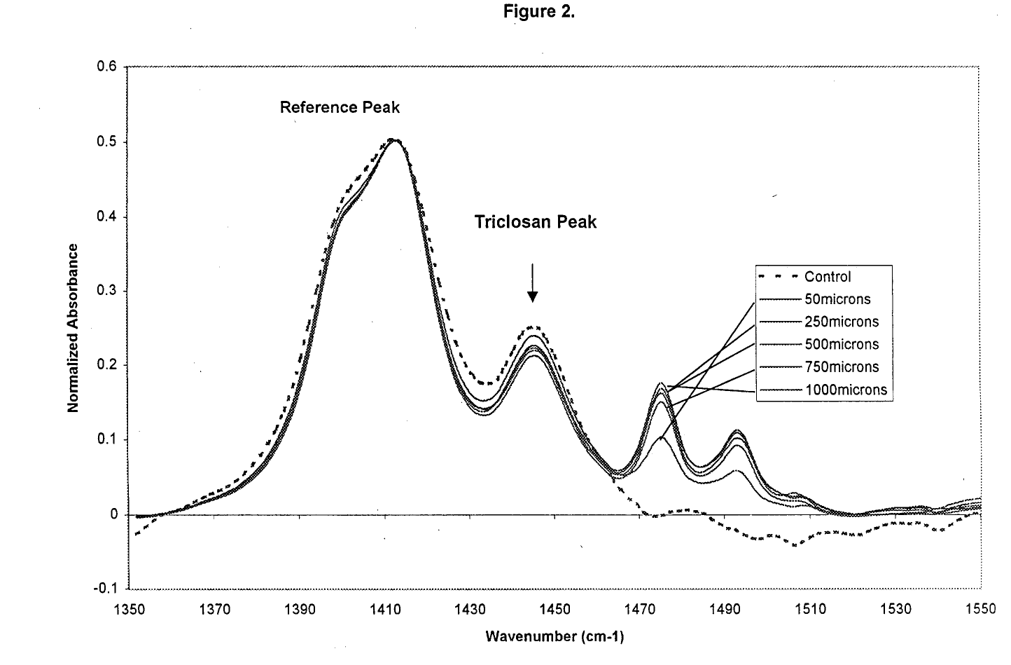

A sample of LSR60HS, a platinum-cured, two-part silicone elastomer, from Applied Silicone, Ventura, Calif., was mixed in a Kitchen Aid® mixer under vacuum to avoid trapped air. The mixture was compression molded in a pre-heated Carver press at 240° F. for 10 minutes. The silicone rubber was then post-cured at 410° F. for 2 hours. This sample, which contains no triclosan, was used as the control in FIGS. 1 and 2.

### Comparative Example IX

LSR60HS, a platinum-cured, two-part silicone elastomer, from Applied Silicone, Ventura, Calif., and 0.25 weight % triclosan was mixed in a Kitchen Aid® Mixer under vacuum to avoid trapped air. The mixture was compression molded in a pre-heated Carver press at 240° F. for 10 minutes. The silicone rubber was then post-cured at 410° F. for 2 hours. This sample was microtomed and analyzed by FT-IR as described in Example II. FT-IR data presented in FIG. 2 clearly shows the homogeneous distribution of the triclosan agent distributed within the polymer matrix.

### Comparative Examples X-XII

Three samples of Vialon™ polyurethane available from Becton Dickinson and Company, Franklin Lakes, N.J. were prepared by melt blending. A first sample was prepared by extruding chips of Vialon™ polyurethane through an extruder at a temperature between about 320° F. and 380° F. Two other samples were prepared in the same manner, one sample containing 3 weight percent triclosan, and the third sample containing six weight percent triclosan.

After impregnating polymers with antimicrobial agent, it is useful to determine the rate at which a polymer can release the antimicrobial agent into the environment. To test for this property, the samples made in example IV and comparative examples X-XII were then exposed to aqueous phosphate buffer solution, and the release of triclosan from the polymeric samples was monitored over a period of days using high pressure liquid chromatography (HPLC). The triclosan release data is shown in FIGS. 3 and 4. The data in FIGS. 3 and 4 clearly shows that the sample made by the inventive method releases triclosan into the phosphate buffer solution at a much greater rate than the melt blended samples prepared according to comparative examples X-XII. This higher triclosan release rate is indicative that medical devices produced according the method of the invention will have greater antimicrobial activity than samples produced by the melt blending method.

The method of the invention can be used to impart antimicrobial properties to a wide variety of medical devices. For example, catheters, catheter connectors, tracheal tubes, shunts, ventilators tubes and like devices can be impregnated with triclosan according to the invention. However, the invention is not limited to any particular device and may include other devices useful in consumer healthcare, such as sterile packaging and personal hygiene products.

According to the present invention, samples containing greater than 5 parts per million of triclosan in the polymeric material were obtained. As illustrated in FIGS. 1-2, the method of this invention provides for a polymer substrate with a higher concentration of antimicrobial agent at the surface relative to the bulk substrate. As shown in FIGS. 3-4, this non-homogenous distribution of agent provides substantial improvement of available triclosan when the polymer substrate is exposed to aqueous media as compared to melt blending. Thus, a more effective anti-infective product is obtained than otherwise would be possible.

The concentration and depth of the triclosan in a polymeric medical device can be controlled by varying the concentration of the triclosan in the solution and contact time of the solution with the polymeric medical device, which can be determined by experimentation.

It will be apparent to those skilled in the art that various modifications and variations can be made to the present invention without departing from the spirit or scope of the invention. Thus, it is intended that the present invention cover the modifications and variations of this invention provided they come within the scope of the appended claims and their equivalents.

What is claimed is:

1. A method of impregnating a polymeric medical device with triclosan comprising the steps of:
dissolving triclosan in a compressed fluid to form a solution;
contacting the polymeric medical device with the solution at a pressure and temperature and for a time sufficient to diffuse the solution into the polymeric medical device; and
separating the solution from the polymeric medical device.

2. The method of claim 1, wherein the compressed fluid is carbon dioxide.

3. The method of claim 2, wherein polymeric medical device is made from a material selected from the group consisting of polyurethane, silicone rubber, polycarbonate, ABS, polypropylene, polyethylene, and polyvinyl chloride.

4. The method of claim 2, wherein the pressure of the solution during the contacting step is maintained above 1500 pounds per square inch.

5. The method of claim 1, wherein the medical device is a catheter.

6. The method of claim 1, wherein the medical device is a connector for a catheter.

7. The method of claim 1, wherein the triclosan is non-homogeneously impregnated into the medical device.

8. The method of claim 8, wherein the concentration of triclosan decreases from the exterior of the device to the interior of the device.

9. A medical device made by the method of claim 1.

10. The medical device of claim 9, wherein the device contains at least 5 parts per million of triclosan.
